(19) United States
(12) Plant Patent Application Publication
(10) Pub. No.: US 2015/0216096 P1
(43) Pub. Date: Jul. 30, 2015

(54) Genie-2, a low profile gardenia

(50) ;

(71) Applicant: Allyn Cook, Gainesville, FL (US)

(72) Inventor: Allyn Cook, Gainesville, FL (US)

(21) Appl. No.: 13/999,182

(22) Filed: Jan. 27, 2014

(51) Int. Cl. A01H 5/00

(52) U.S. Cl. PLT/255

(57) ABSTRACT

The plant herein described and named Genie-2 is physically unique and readily distinguished by the size and shape of leaves in combination with maintenance of an exceptionally low foliar profile and prolific production of flowers with the recognizable fragrance of gardenia.

### BACKGROUND OF THE INVENTION

About 1750, Dr. Alexander Garden (+/−1730-1791), an immigrant medical practitioner and amateur botanist found a plant in New York state that he could not identify. Following a lengthy debate with two renowned taxonomists about proper naming of this plant and characterization of a “new” plant genus, the matter was resolved when, in London in 1760, a man named Ellis identified the plant as Gardenia jasminoides Linn. The naming was intended to honor Dr. Garden for his contribution and to encourage him to continue his valuable field work. Although the cultivar found by Dr. Garden has been lost in time, there was, at the same time, another “unidentified” cultivar of the same plant that had been given a ship captain as he rounded the Cape of Good Hope that was completely ignored at the time of this lengthy debate. What is presumed to be that specific cultivar, and still designated “Cape Jasmine”, can be found in Florida today. A number of gardenia cultivars with “single” or “double” flowers have appeared since time and disappeared for various reasons including inadequate aesthetic value.

Gardenia cultivars with double flowers that are popular today routinely do not produce seed because flowers lack the “functional” parts although some pollen can be found on occasion. Accordingly, the possibility for new cultivars has been severely limited before the discovery of cv Star Bright (pat. 8887) that provided consistent seed production. Combined with capability for seed production and interspecific hybridization (pat. 21,541) populations are possible for selecting individual plants with degrees of multigenic traits(s) such as Star Dust. The utility of Star Bright for breeding purposes has been further extended to accomplish the experimental creation of a new species as defined by successful combination of two interspecific hybrids (unpublished; see ref. 2, p 324). Unfortunately, all 90 plants of the “new species” were short-lived. While it is now theoretically possible to create completely new and different gardenia cultivars, neither plant habit nor flowering capability are included in this catalog. Plants with different or specific character(s), including physical traits, still must be sought through propagation and evaluation of individual seedlings.

### DETAILED BOTANICAL DESCRIPTION

Mature plants of the cultivar may reach more than one meter in diameter but rarely produce foliage more than 0.5 m above ground level. Early branching often is near 45 degrees and branches of significant size are typically strong enough to maintain more than a 35 degree angle above the ground. Few branches droop sufficiently to the touch the ground.

Developing stems of Genie-2 are light green (RHSC 137A) when they first appear. The stems progress though darkening shades of green as they lengthen and increase in diameter until they eventually are decidedly brown by the time they reach 3-5 mm in diameter. Leaves are typically a darker shade of green than in common cultivars of the species and often have a shiny appearance as if they have just been washed clean and waxed. Lead development is confined to nodes with inter-nodal length partially dependent on amount and duration of daily sunlight exposure. Plants grown under low sunlight develop less compact and more open foliage that may reach 2 m in length but the low profile of the entire plant is consistently retained.

Leaves develop in pairs, one from each side of the stem. Length of the lamina may reach 60 mm and typically tapers from both ends so the widest place is closer to the apical end where the width can reach 15 mm. Multiple secondary veins, more prominent from below than above, originate from a straight mid rib and extend forward at an angle to the leaf margin.

Flower buds only develop from nodes and each developing bud rests in a calyx cup that typically shows six “fingers” surrounding a single bud at the end of a short stem (1-2 cm) usually no more than 3 mm in diameter.

Flowers approximately 5 cm each are borne most frequently from the middle of the branch toward the distal end. There are typically three whorls of completely formed petals with attachment and spacing of petals alternately with those of the next upper, smaller whorl. Length and width of individual petals is slightly less with each innermost whorl. In the outermost whorl, petals are 30 mm in length, 20 mm in width with edges irregularly down-curved. Petals in the innermost, fully formed whorl are much reduced (10×13) sometime's with up-curving tips. A mixture of small, aborted and severely reduced reproductive structures are often imprinted at the bases of some of the innermost petals that often are compacted to make a “ball” 3-5 mm in diameter in the center of the flower. There is no physical evidence of reproductive structures despite the common appearance of the colored imprints. All flowers exude the typical and distinctive gardenia fragrance.

Genie-2 plants respond well to attentive care and bear more flowers of better quality if “spent” flowers are routinely removed as they decline.

Multiplication of Genie-2 is readily accomplished both by rooting cuttings and by tissue culture. The distinctive physical characters that identify the cultivar are retained though both procedures as is the quality and quantity of the fragrance.

For colors cited herein see The Royal Horticultural Society Chart, 2001 edition.

### EXPLANATION OF ILLUSTRATIONS

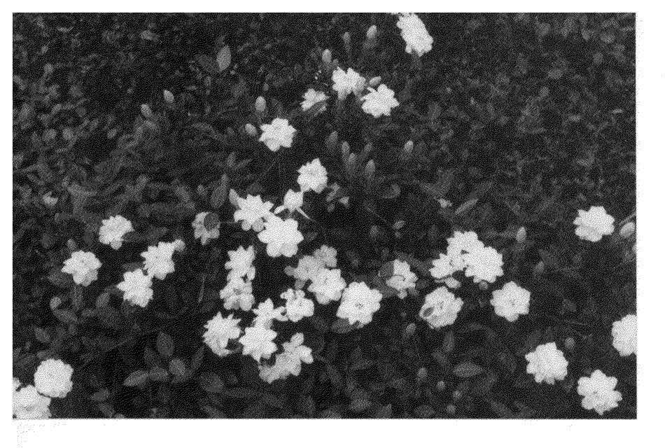

FIG. 1. Genie-2 plant grown in 60% sun over ground cover of Carolina Jasmine.

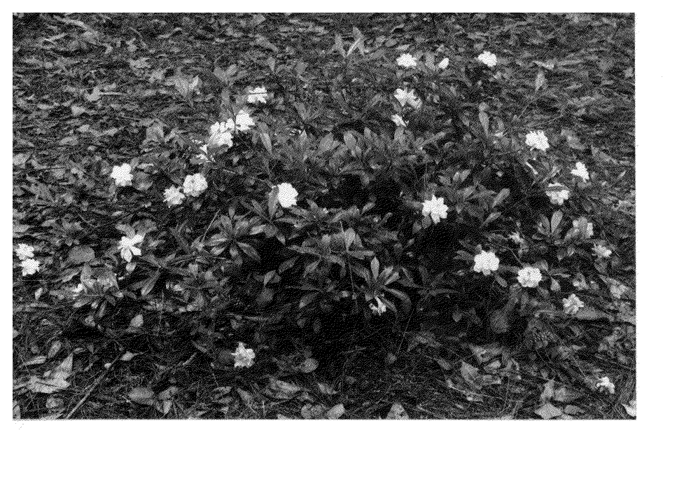

FIG. 2. Genie-2 plant grown in 40% sun.

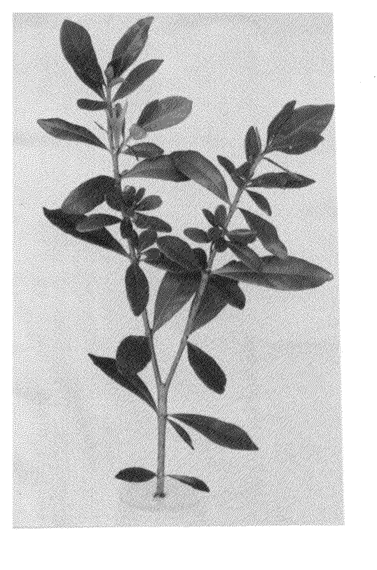

FIG. 3. Small branch showing leaf orientation and placement.

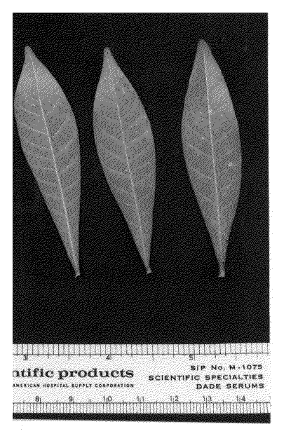

FIG. 4. Mature leaves (underside) showing size, shape, and venation.

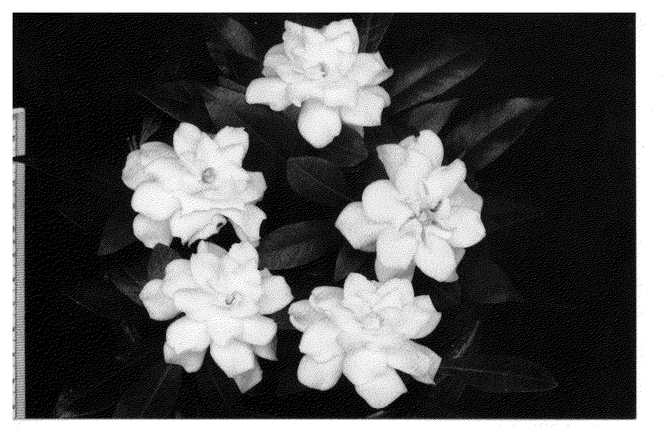

FIG. 5. Genie-2 flowers some showing (non-functional) imprints.

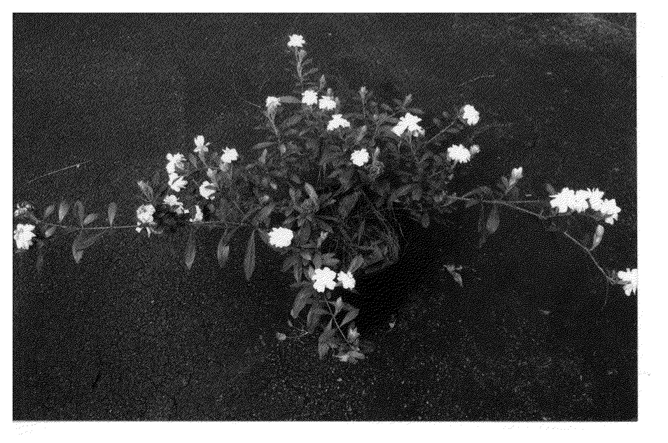

FIG. 6. Original, potted Genie-2 plant grown in full (75%) sun.

What is claimed is:

1. A new and distinct cultivar of gardenia named ‘Genie-2’ as described herein and formally claimed by the finder.
